(19) United States
(12) Patent Application Publication
(10) Pub. No.: US 2015/0335543 A1
(43) Pub. Date: Nov. 26, 2015

(54) Laponite Clay in Cosmetic and Personal Care Products

(71) Applicant: Avon Products, Inc., Suffern, NY (US)

(72) Inventors: Ashley L. Howell, Oakland, NJ (US); Candice DeLeo Novack, Monroe, NY (US); Amanda M. Stagg, Morris Plains, NJ (US); Stavroula Patel, Pleasantville, NY (US); Emily H. Rubinson, Suffern, NY (US)

(73) Assignee: Avon Products, Inc., Suffern, NY (US)

(21) Appl. No.: 14/282,311

(22) Filed: May 20, 2014

(51) Int. Cl. A61K 8/25; A61K 8/81; A61Q 1/02; A61K 8/73

(52) U.S. Cl. CPC A61K 8/25; A61K 8/731; A61K 8/8111; A61Q 1/02; A61K 2800/48; A61K 2800/43

(57) ABSTRACT

The invention relates generally to formulations having one or more colorants segregated from other colorants in the same formulation. The formulations are typically for topical application to an integument.

### FIELD OF INVENTION

The invention relates generally to formulations having one or more colorants segregated from other colorants in the same formulation. The formulations are typically for topical application to an integument.

### BACKGROUND

Traditional color cosmetics contain colorants, such as pigments, lakes, and dyes, that are selected to provide certain color attributes (e.g., hue value and chroma) when applied to the skin. Such products are limited to providing a uniform appearance on skin because the colorants are homogeneously blended in the formulation. There is a need for compositions that are capable of delivering colorants to the skin in an inhomogeneous manner in order to increase the range of effects that can be achieved.

Laponite clays are synthetic, layered silicates known in the art as additives to cosmetics and personal care products such as foundations, sunscreens, and toothpaste, for their ability to act as thickening agents, gelling agents and fillers (see, e.g., U.S. Pat. Nos. 8,603,444; 8,586,011; 8,524,203; and 8,506,953, the disclosures of which are hereby incorporated by reference). However, heretofore, laponite clays have not been used to segregate discrete ingredients (e.g., colorants) within a single formulation. It is therefore an object of the present invention to provide compositions useful in cosmetics and personal care products that comprise laponite clays to segregate two or more colorants within the composition, and which are capable of delivering the two or more colorants in a single formulation that do not blend substantially prior to application, and that may remain segregated even after application to a human integument, or which may be blended together after application to an integument.

The foregoing discussion is presented solely to provide a better understanding of nature of the problems confronting the art and should not be construed in any way as an admission as to prior art nor should the citation of any reference herein be construed as an admission that such reference constitutes “prior art” to the instant application.

### SUMMARY OF THE INVENTION

In accordance with the foregoing objectives and others, the present invention provides laponite-clay-containing formulations having one or more ingredients (e.g., colorants) segregated or separated from other constituents of the same formulation, and methods for preparing them. It has surprisingly been found that some ingredients in a cosmetic formulation can be effectively shielded from other ingredients within the same formulation when such formulations are prepared according to the present invention. The present invention provides methods for making compositions comprising a plurality of phases, at least two of which have different optical attributes from one another. The optical attribute may be hue, chroma, color travel, iridescence, sparkle etc. The compositions may have a multi-colored appearance (e.g., a speckled appearance) prior to application to a human integument, and may be blended together after application to a human integument (e.g., by rubbing) to provide a uniform appearance or a non-uniform appearance depending on the degree of blending and/or thickness of the clay shield surrounding the pigment phase. Alternatively, the compositions may maintain the optical attribute following application to a human integument to purposely provide a speckled appearance.

Without wishing to be bound by any particular theory, it is believed that the compositions of the invention have the general form of liquid droplets or globules (e.g., a discontinuous phase) suspended within another liquid (e.g., a continuous phase), wherein discs of laponite clay surround the droplets and are held at the interface between the liquid droplets (e.g., in predominantly edge-to-edge orientation) and the suspending liquid by a thickener (typically polymeric) dissolved in the liquid droplets. This is believed to effectively provide a shell surrounding the liquid droplets which prevents or retards migration of components, including colorants, across the interface. The use of the terms “continuous” and “discontinuous” phases is not to be construed as an emulsion, unless otherwise indicated. The shear (e.g., by rubbing) encountered by the compositions during application is believed to release the contents of the discontinuous phase into the continuous phase. Once released, the contents of the discontinuous phase(s) may blend together with the continuous phase. However, in some embodiments, the contents of the discontinuous phase(s) are not released on application, and do not blend together with the continuous phase, for example providing an inhomogeneous color deposit on an integument.

Generally, the methods of the invention comprise mixing together (e.g., under low shear or under high shear) a first liquid composition (the continuous phase) comprising water and a laponite clay, and a second liquid composition (the discontinuous phase) comprising water and a polymeric thickener, so that the discontinuous phase forms droplets that are suspended in the continuous phase. The laponite clay (e.g., lithium, magnesium, and sodium silicate; lithium, magnesium, sodium silicate, and tetrasodium pyrophosphate; sodium, magnesium, and fluorosilicate; or sodium, magnesium, fluorosilicate, and tetrasodium pyrophosphate) may comprise between about 0.1% to about 20% (e.g., from about 5% to about 8%) by weight of the total composition. The polymeric thickener (e.g., an acrylate polymer or cellulosic thickener (but not polyethylene glycol) comprises between about 0.1% to about 10% (e.g., from about 0.05% to about 2%) by weight of the total composition). It has been found that colorants (e.g., those used in cosmetic or personal care formulations, including, for example, pigments, lakes, dyes, pearlescents, diffracting pigments, opacifying fillers, goniochromatic coloring agents, etc.) can be incorporated into one or both of the phases and are effectively shielded from other colorants in the same formulation. Therefore, the first and second liquid compositions (and any other number of liquid compositions) may have different optical properties (e.g., hue value, chroma, color travel, interference, reflectance, etc.) For example, if a colorant is added to the second liquid composition (discontinuous phase), the colorant will remain in the droplets rather than mixing with the first liquid composition (continuous phase) giving an inhomogeneous or speckled appearance to the composition. Alternatively, a colorant incorporated into the first composition (continuous phase), will be shielded and not mix with the colorants or other components that are in the droplets of the second liquid composition (discontinuous phase). Therefore, it has surprisingly been found that a plurality of colorants can be delivered in the same formulation (e.g., in the form of a cream, a lotion, or a solid stick) and remain segregated prior to and/or after application to a human integument if the colorants are separated as described (e.g., one colorant in a droplet of the discontinuous phase and one colorant in the continuous phase). The interaction between the colorants in the continuous and discontinuous phase is eliminated, minimized, retarded, etc. The colorants used in any two or more of the liquid compositions may be the same colorants, but used at different levels, to effect a different optical attribute (e.g., different amounts of a same pigment to produce different shades of the same color). Alternatively, one or more of the phases may be free of colorants. In another variant of the invention, the discontinuous phase can be prepared from two or more different liquid compositions, each having different colorants (e.g., one liquid composition comprising water and a thickener can have a first colorant, and another liquid composition comprising water and a thickener can have a second colorant, or one liquid composition may have a first colorant and the other lacks any colorant). The colorants may be, for example, pigments, lakes, dyes, pearlescents, interference pigments, glitters, or any other ingredient that can impart an optical attribute to a cosmetic or personal care formulation. Optical materials include any component that alters an optical attribute, including opacifying fillers and other particulates. Each of the liquid compositions of the discontinuous phase can be separately added with stirring to the continuous phase to form discrete droplets of the first and second liquid compositions, respectively. In this embodiment too, it has been surprisingly found that interaction between the colorants is eliminated, minimized, retarded, etc.

In another aspect of the invention, compositions are provided comprising a first liquid composition (the continuous phase) that comprises water and a laponite clay, and a second liquid composition (the discontinuous phase) that comprises water and a polymeric thickener, so that the discontinuous phase forms droplets that are suspended in the continuous phase. The laponite clay (e.g., lithium, magnesium, and sodium silicate; lithium, magnesium, sodium silicate, and tetrasodium pyrophosphate; sodium, magnesium, and fluorosilicate; or sodium, magnesium, fluorosilicate, and tetrasodium pyrophosphate) may comprise between about 0.1% to about 20% (e.g., from about 5% to about 8%) by weight of the total composition. The polymeric thickener (e.g., an acrylate polymer or cellulosic thickener (but not polyethylene glycol) comprises between about 0.1% to about 10% (e.g., from about 0.05% to about 2%) by weight of the total composition). It has been found that colorants (e.g., those used in cosmetic or personal care formulations, including, for example, pigments, lakes, dyes, pearlescents, diffracting pigments, opacifying fillers, goniochromatic coloring agents, etc.) can be incorporated into one or both of the phases and are effectively shielded from other colorants in the same formulation. Therefore, the first and second liquid compositions (and any other number of liquid compositions) may have different optical properties (e.g., hue value, chroma, color travel, interference, reflectance, etc.) For example, if a colorant is added to the second liquid composition (discontinuous phase), the colorant will remain in the droplets rather than mixing with the first liquid composition (continuous phase) giving an inhomogeneous or speckled appearance to the composition. Alternatively, a colorant incorporated into the first composition (continuous phase), will be shielded and not mix with the colorants or other components that are in the droplets of the second liquid composition (discontinuous phase). Therefore, it has surprisingly been found that a plurality of colorants can be delivered in the same formulation (e.g., in the form of a cream, a lotion, or a solid stick) and remain segregated prior to and/or after application to a human integument if the colorants are separated as described (e.g., one colorant in a droplet of the discontinuous phase and one colorant in the continuous phase). The interaction between the colorants in the continuous and discontinuous phase is eliminated, minimized, retarded, etc. The colorants used in any two or more of the liquid compositions may be the same colorants, but used at different levels, to effect a different optical attribute (e.g., different amounts of a same pigment to produce different shades of the same color). Alternatively, one or more of the phases may be free of colorants. In another variant of the invention, the discontinuous phase can be prepared from two or more different liquid compositions, each having different colorants (e.g., one liquid composition comprising water and a thickener can have a first colorant, and another liquid composition comprising water and a thickener can have a second colorant, or one liquid composition having a first colorant and the other lacking any colorant). The colorants may be, for example, pigments, lakes, dyes, pearlescents, interference pigments, glitters, or any other ingredient that can impart an optical attribute to a cosmetic or personal care formulation. Optical materials include any component that alters optical attributes, including opacifying fillers and other particulates. Each of the liquid compositions of the discontinuous phase can be separately added with stirring to the continuous phase to form discrete droplets of the first and second liquid compositions, respectively. In this embodiment too, it has been surprisingly found that interaction between the colorants is eliminated, minimized, retarded, etc. Accordingly, another aspect of the invention provides methods for delivering in a single formulation, one or more colorants or other ingredients that can impart color or other optical properties (e.g., hue value, chroma, color travel, interference, etc.) to a cosmetic or personal care formulation. The method comprises applying (e.g., by rubbing or blotting, etc.) to a human integument (e.g., skin or hair, such as skin of the face or hands; hair on the head; eyelashes; or eyebrows, etc.) a composition of the invention.

In some embodiments, a poly(alkylene oxide), such as polyethylene glycol (PEG) effective to disrupt the laponite shell surrounding the discontinuous phase droplets may be brought into contact with the discontinuous phase droplets to promote mixing of their contents in the presence of shear (e.g., rubbing the composition onto the skin). In one embodiment the poly(alkylene oxide) comprises PEG-12 carnauba wax. In another embodiment, the poly(alkylene oxide) comprises PEG-40. In some embodiments the poly(alkylene oxide) is present in one or more of the first, second and/or third liquid compositions. In another embodiment a poly(alkylene oxide) is applied to a human integument prior to of after application of a multi-domain composition according to the invention, followed by rubbing to bring the poly(alkylene oxide) into contact with the discontinuous phase droplets and to release and blend their contents with the remainder of the composition. In some embodiments, the composition of the invention will achieve a color change after rubbing in the presence of poly(alkylene oxide), such as PEG.

These and other aspects of the present invention will become apparent to those skilled in the art after a reading of the following detailed description of the invention, including the appended claims.

### DETAILED DESCRIPTION

All terms used herein are intended to have their ordinary meaning unless otherwise provided. All ingredient amounts provided herein are by weight percent of the total composition unless otherwise indicated.

By “topically acceptable” is meant that an ingredient is generally regarded as safe and non-toxic for application to a human integument.

By “colorant” is meant pigments, lakes, dyes, pearlescents, interference pigments or fibers, glitters, particles with a metallic tint, metallic flakes, glass microspheres and plates, goniochromatic coloring agents (e.g., multilayer interference structures and liquid-crystal coloring agents), diffracting pigments, fibrous pigments, optical brighteners, thermochromic agents, opacifying fillers, and any ingredient that is capable of imparting color or other optical attributes to a cosmetic or personal care formulation.

It has surprisingly been found that components, including colorants, of a cosmetic or personal care formulation can be effectively shielded or segregated from other components, including colorants, within the same formulation when such formulations are prepared according to the methods of the present invention. As a result, more than one colorant (or the same colorant present in different amounts) can be delivered in the same formulation without spontaneous mixing, thereby imparting an inhomogeneously colored appearance (e.g., speckled) to a cosmetic or personal care formulation. The inhomogeneous appearance may remain after application to a human integument, or the different colorants may be blended together after application to a human integument (e.g., by rubbing) to provide a substantially uniform appearance. The methods generally comprise preparing a composition by mixing together a first liquid composition, comprising water and a laponite clay (continuous phase), and adding to the first composition, by mixing (or stirring), a second liquid composition, comprising water and a polymeric thickener (discontinuous phase). Without wishing to be bound by theory, it is believed that the discontinuous phase forms droplets that are suspended in the continuous phase. Colorants such as those used in cosmetics and personal care formulations can be incorporated into the continuous phase, the discontinuous phase, or both phases. When the discontinuous phase is added to the continuous phase, the components in the discontinuous phase remain in the droplets of the discontinuous phase, rather than mixing with the continuous phase.

In the compositions of the invention, each colorant in the discontinuous phase is effectively shielded or separated from the continuous phase, so that the interaction between or among them is eliminated, reduced, or retarded compared to an otherwise identical composition lacking laponite clay. In addition, each colorant in the discontinuous phase is effectively shielded or separated from every other colorant in the discontinuous phase, so that the interaction between or among them is eliminated, reduced, or retarded compared to an otherwise identical composition lacking laponite clay. In some embodiments, it is believed that shear (e.g., by rubbing or pressure) that is encountered by the compositions during application, for example, to a human integument, may release the contents of the discontinuous phase into the continuous phase. Once released, the contents of the discontinuous phase may blend together with the continuous phase. However, in some embodiments, the components are not released from the discontinuous phase, and the contents do not blend together with the continuous phase, thereby providing an inhomogeneous color deposit on an integument.

The first liquid composition (continuous phase) in the compositions of the invention comprises water and at least one laponite clay. Laponite clay refers generally to a synthetic, layered silicate clay that has a layer structure in the form of disc-shaped crystals when dispersed in water. Macromolecules of the laponite clay have a disc-shaped crystal similar to bentonite and hectorite, but are more than one order of magnitude smaller in size. In contrast to clays with a large (e.g., greater than 100) aspect ratio (ratio of length to height) that tend to aggregate in a face-to-face lamellar fashion, laponite clay tends to form partially delaminated disordered aggregates through edge-to-edge interactions. Laponite clay may have, without limitation, an aspect ratio of between about 10 and about 80, or between about 15 and about 50, or between about 20 and about 40, or between about 25 and about 35. In one embodiment, the laponite clay has an aspect ratio of about 30. It will be understood that these aspect ratios represent averages. The surface of the crystal may have a negative charge of about 50-55 mmol·100 g⁻¹. The edges of the crystal may have small, localized positive charges that are about 4-5 mmol·100 g⁻¹. Laponite clay may have a physical surface area over about 900 m²·g⁻¹. Laponite clay may comprise about 8% by weight water that may be released from its crystal structure at temperatures above about 150° C. Commonly known laponite clays encompassed by the invention include without limitation those that comprise: lithium, magnesium, and sodium silicate; those that comprise lithium, magnesium, sodium silicate, and tetrasodium pyrophosphate; those that comprise sodium, magnesium, and those that comprise fluorosilicate; and sodium, magnesium, fluorosilicate, and tetrasodium pyrophosphate. In some embodiments, laponite clay may comprise one or more of SiO₂, MgO, Na₂O, and LiO₂. In one embodiment, laponite clay may comprise from about 60 to about 70% (w/w) SiO₂, and/or from about 20 to about 40% (w/w) MgO, and/or from about 1 to about 5% (w/w) Na₂O, and/or from about 0.1 to about 3% (w/w) LiO₂. In some embodiments, laponite clay comprises SiF₆⁻². In some embodiments, laponite clay comprises from about 10 to about 70%, or from about 50 to about 70% SiF₆⁻². Any combination of two or more of these laponite clays may also be used in the multi-component compositions of the invention.

In one embodiment, the diameter of the disc-shaped crystal in the laponite clay may be from about 1 nm to about 300 nm, from about 5 nm to about 150 nm, or from about 10 nm to about 100 nm. In one embodiment, the diameter of the disc-shaped crystal is from about 15 nm to about 50 nm. In another embodiment, the diameter of the disc-shaped crystal is from about 20 nm to about 40 nm, or from about 25 nm to about 35 nm, or about 25 nm, or about 35 nm.

In one embodiment, the thickness of the disc-shaped crystal in the laponite clay may be from about 0.1 nm to about 3 nm, from about 0.5 nm to about 1.5 nm, or from about 0.8 nm to about 1.2 nm. In one embodiment, the thickness of the disc-shaped crystal is from about 0.8 nm to about 1 nm. In another embodiment, the thickness of the disc-shaped crystal is about 0.92 nm. In one embodiment, the density of laponite clay is between about 2.5 and about 2.55 gm/cm³, or about 2.53 gm/cm³.

Non-limiting examples of laponite are disclosed in Herrera et al., J. Mater. Chem., 2005, 863-871; and Cummins, J. Non-Crystalline Solids, 2007, 353, 3891-3905, the disclosures of which are hereby incorporated by reference. Suitable laponites are available from Rockwood Additives as either gel-forming or sol-forming grades. Gels and sols comprising laponites may be thixotropic and/or shear thinning Special mention may be made of the sol forming grade S482.

The amount of laponite clay in the multi-component composition is not particularly limited. For example, the laponite clay may comprise between about 0.01% to about 40% by weight of the total composition. In other embodiments, the laponite clay comprises between about 0.03% and about 30%, between about 0.05% and about 25%, or between about 0.1% and about 20% by weight of the total composition. In some preferred embodiments, the laponite clay comprises between about 0.5% and about 10%, between about 1.0% and about 5.5%, or between about 2% and about 4% by weight of the total composition. In some embodiments, the laponite clay comprises between about 4% and about 10% or between about 5% and about 8% of the total composition.

In some embodiments, the first liquid composition (continuous phase) further comprises a rheological additive, such as the rheological additive having the INCI name Bentonite (and) Xanthan Gum (available as Optigel WX from BYK Additives and Instruments).

The second liquid composition and any additional liquid compositions comprising the discontinuous phase (e.g., third, fourth, fifth liquid compositions, etc.) in the compositions of the invention may comprise water and at least one polymeric thickener.

In some embodiments, the second and third liquid compositions are different from one another. In some embodiments, all of the liquid compositions are different from one another. In other embodiments, all of the liquid compositions comprising the discontinuous phase are different from one another.

The polymeric thickener may be the same or different in each liquid composition of the discontinuous phase, and may comprise, for example, one or more of acrylates copolymers, carboxyvinyl polymers, stearic acid, fatty alcohols, such as cetyl alcohol, stearyl alcohol, carbomers, myristyl stearate, cetyl stearate, magnesium aluminum silicate, polyacrylamide/isoparaffin/laureth-7 (Seppigel), hydroxyethyl cellulose, propylene glycol monostearate, or cellulosic thickeners, including hydroxypropyl cellulose, carboxymethyl cellulose, hydroxyalkyl cellulose, carboxymethylcellulose, and vegetable gums such as xanthan gum and locust bean gum.

Acrylic polymers that may be used as a polymeric thickener include pure acrylic polymers (such as methylmethacrylate and alkyl acrylates, such as butyl acrylate); styrene-acrylic (such as based on styrene and alkyl acrylates, for example, 2-ethylhexyl-acrylate); vinyl acetate copolymers with alkyl acrylate or ethylene or maleates; and styrene-butadiene based polymers, and the like. More particularly, the polymer preferably contains two or more monomers selected from styrene, methylmethacrylate, vinyl acetate, butadiene, n-butyl acrylate, 2-ethylhexyl-acrylate, ethylacrylate, methylacrylate, isopropylacrylate, vinyl propionate, dibutyl maleate, ethylene, tert-butyl acrylate, methacrylic acid, acrylic acid, acrylamide, methacrylamide, and hydroxyethyl methacrylate.

In some embodiments, preferred acrylate polymer thickeners comprise high molecular weight acrylate polymers and acrylate copolymers.

In one embodiment, the polymeric thickener comprises hydroxyl ethylcelluose. In another embodiment, the polymeric thickener comprises an acrylate polymer, for example, acrylates/C10-30 alkyl acrylate crosspolymer. In yet another preferred embodiment, the polymeric thickener comprises a carboxyvinyl polymer, for example, Carbopol 940.

The amount of the polymeric thickener in the multi-component compositions is not particularly limited, and may comprise, in some embodiments, between about 0.01% and about 25% by weight of the total composition. In other embodiments, the polymeric thickener may contain between about 0.01% and about 20%, between about 0.5% and about 15%, between about 1% and about 10%, between about 1% and about 8%, between about 1% and about 6%, between about 1% and about 4%, between about 1% and about 2%, or between about 0.5% and about 1% by weight of the total composition. In some embodiments, the polymeric thickener may contain between about 0.1% and about 2.0%, or between about 0.05% to about 10% by weight of the total composition.

Poly(alkylene oxides), such as polyethylene glycol (PEG), have been found to promote the breakdown of the discontinuous phase droplets on rubbing or shearing and consequently, allow their contents to be released into the continuous phase. This effect may be advantageously used to allow for controlled mixing of the discontinuous phases. For example, by incorporating an poly(alkylene oxide), such as PEG, into each of the discontinuous phases (e.g., the second and third liquid compositions), the contents of those droplets are realeased when the product is rubbed onto the skin and the two different optical attributes of those droplets (e.g., two different colors) are blended to form a homogenous film or layer on the skin that has an optical property resulting from the blending of the two differently colored discontinuous phases. The controlled blending can be used to enhance the range of cosmetic effects that can be produced on the skin. In some embodiments, the controlled blending may be used to affect a color change. This may be useful, for example, when the composition is formulated as a sunscreen so the user knows when the product has been uniformly applied to the skin by the changed color (or disappearance of color) when the composition is rubbed sufficiently onto the skin.

In some embodiments, the poly(alkylene oxide) containing ingredient may be applied to the skin before or after the compositions of the invention are applied. For example, the user may first apply a multi-component composition of the invention to the skin and subsequently apply an overcoat of a poly(alkylene oxide) containing composition followed by rubbing to release the color components from the laponite-encased discontinuous phases. Alternatively, the poly(alkylene oxide) containing ingredient may be applied as a base coat and the compositions of the invention subsequently applied over top of the base coat. In other embodiments, the product may be formulated to segregate a poly(alkylene oxide) containing ingredient from the compositions of the invention but allow them to be simultaneously deposited on the skin. For example, a “core” and “shell” type lipstick product may have a solid core surrounded by a solid shell surrounding the core. Either the shell or the core could comprise the multi-domain compositions of the invention, while the other core or shell component would comprise an poly(alkylene oxide) containing ingredient, such as PEG. On application to the lips, the core and shell compositions are blended, bringing the PEG into contact with the laponite-encased discontinuous phases to promote mixing and blending of the colorant components. In one embodiment, the core of core and shell lipstick will comprise PEG, for example in an amount from about 1-99% by weight, or from about 2.5-90% by weight, or from about 5-75% by weight, or from about 10-50% by weight, or from about 15-35% by weight of the inner core. As one skilled in the art will recognize, there are numerous variations on the core and shell configuration that are also contemplated, including molded sticks wherein half of the stick comprises the multi-domain composition of the invention and the other half comprises a PEG component.

It is contemplated that any poly(alkylene oxide) containing ingredient may be used to affect the breakdown of the discontinuous phases. For example, poly(alkylene oxide) emulsifiers, such as polysiloxanes having poly(alkylene oxide) side chains (e.g., PEG and/or PPG) may enhance rupture and mixing of the discontinuous phases. Pegylated waxes, such as PEF-12-carnuaba wax may also be used for this purpose. Special mention may be made of PEG oils and waxes, such as PEG-40.

In some embodiments, the thickener may comprise PEG. For example, the thickener in the second and/or third liquid composition may include PEG, for example, PEG-40. In other embodiments, the polymeric thickener does not comprise a polyalkylene oxide such as polyethylene glycol (PEG), or comprises less than about 1% by weight, or less than about 0.5% by weight, or less than about 0.1% by weight of such polyalkylene oxides. In some embodiments, the composition does not comprise a polyalkylene oxide such as PEG, or comprises less than about 1% by weight, or less than about 0.5% by weight, or less than about 0.1% by weight of such polyalkylene oxides.

In preparing the compositions of the invention, a colorant may be added to the discontinuous phase, so that it remains in the droplets of the discontinuous phase rather than mixing with the continuous phase. For example, the composition may comprise a first liquid composition comprising water and a laponite clay, to which is added, with stirring, a second liquid composition comprising water, a polymeric thickener, and a colorant. The component is shielded from the continuous phase so that that the interaction or mixing of the colorant and the continuous phase is eliminated, reduced, or retarded compared to an otherwise identical composition lacking the laponite clay.

In another embodiment, a colorant is added to the continuous phase, and the colorant does not mix with the colorants that are in the droplets of the discontinuous phase. For example, the compositions of the invention may comprise a first liquid composition comprising water, a laponite clay, and a first colorant (continuous phase), to which is added, with stirring, a second liquid composition (discontinuous phase) that comprises water, a polymeric thickener, and a second colorant. The first and second colorants are shielded from each other such that the interaction between them is eliminated, reduced, or retarded compared to an otherwise identical composition lacking the laponite clay.

In other embodiments, the discontinuous phase may be prepared from two or more liquid compositions, each liquid composition comprising a different colorant, and each liquid composition being separately added (with stirring) to the continuous phase. All of the colorants in the continuous phase (if any) and the discontinuous phase are shielded from one another such that the interaction among them is eliminated, reduced, or retarded compared to an otherwise identical composition lacking the laponite clay.

The colorants that are separated or shielded from one another may be different colorants, or they may be the same colorants present at different levels, thereby imparting different optical attributes, such as, for example, a different shade, hue value, intensity, saturation, reflectance, radiance, etc. than any one or more of the other colorants in the composition. For example, in one embodiment, at least two of the liquid compositions each comprise a different colorant (e.g., pigment). In other embodiments, at least two of the liquid compositions each comprise the same colorant (e.g., pigment), albeit at different levels, such that the optical attributes of the at least two liquid compositions are different. In some embodiments, the compositions provide a plurality of optical attributes.

In some embodiments, at least one of the liquid compositions is lacking a colorant.

In some embodiments, at least two of the liquid compositions each have different optical attributes. In other embodiments, all of the liquid compositions have different optical attributes.

In one embodiment, at least one of the liquid compositions comprises a pigment, and at least one of the liquid compositions comprises an opacifying filler or glitter.

In one embodiment, at least one of the liquid compositions comprises a pigment, and at least one of the liquid compositions comprises a pearlescent.

In some embodiments, the composition comprises more than one color, the colors remaining segregated prior to and/or after application to a human integument. The composition may have a speckled, spotted, or multi-color appearance both prior and/or after application to a human integument.

In other embodiments, application (e.g., by rubbing, blotting, or pressing, etc.) of the composition to a human integument may cause blending of the colorants to provide a substantially uniform appearance on the human integument (e.g., providing an inhomogeneous color deposit). The blending of colorants may result in a change in optical attributes, such as a change in color, a change in hue, a change in shade, a change in intensity, a change in saturation, a change in reflectance, a change in radiance, etc., of the composition. The color change may be effected by a physical blending of colorants, a change in pH, a chemical reaction (e.g., oxidation of a two-part reactive dye), or any other mechanism.

The compositions of the invention are preferably capable of maintaining separation between two or more colorants. The colorants in the composition are separated such that the interaction between or among them is reduced, minimized, retarded, etc., compared to an otherwise identical composition lacking laponite clay. Another aspect of the invention provides compositions capable of maintaining separation between one or more colorants in the composition and the continuous phase. The colorants are separated such that the interaction between or among them is reduced, minimized, retarded, etc., compared to an otherwise identical composition lacking laponite clay. Such minimized reduction may yield both unique visual appearance of compositions and better stability of individualized pigmented area (e.g. prevention of shade shifting, pigment bleed, etc. as between different pigmented areas of a composition).

The compositions of the invention may be used in any kind of cosmetic or personal care formulation that can be applied to a human integument. The composition may be added to any suitable cosmetic formulation or personal care product formulation, whether in the form of a liquid, a cream, a lotion, a solid stick, etc.

For example, the cosmetic composition may be, without limitation, in the form of lipstick, lip color, lip gloss, nail polish, foundation, concealer, blush, eye shadow, eye liner, mascara, bronzer, and the like. The personal care product may be, for example, in the form of day creams or lotions, night creams or lotions, sunscreen lotions, sunscreen creams, sunscreen sprays or oils and other SPF products, moisturizers, salves, ointments, gels, body milks, artificial tanning compositions, shampoos, conditioners, fragrances, and the like.

In one embodiment, the compositions of the invention are added to a sunscreen formulation that is in the form of a cream, a spray, or a lotion. In another embodiment, the composition is added to a solid stick formulation (e.g., a lipstick or a foundation stick), which comprises both an inner core and an outer core, the outer core comprising a composition of the invention, comprising one or more colorants (in addition to, for example, any suitable waxes described herein) and the inner core comprising, for example, PEG. In yet another embodiment, the multi-component composition is added to a solid stick formulation (e.g., a lipstick or a foundation stick), which comprises both an inner core and an outer core, the inner core comprising the a composition of the invention, comprising one or more colorants (in addition to, for example, any suitable waxes described herein) and the outer core comprising, for example, PEG. Upon application to a human integument, the one or more colorants may be released from the discontinuous phase, and may blend together to provide a substantially uniform appearance on the human integument (e.g., providing an inhomogeneous color deposit). The blending of colorants may result in a change in optical attributes, such as a change in color, a change in hue, a change in shade, a change in intensity, a change in saturation, a change in reflectance, a change in radiance, etc., of the composition.

The colorants shielded in the compositions of the invention may be any material used in a cosmetic or personal care formulation, and particularly colorants or other ingredients that can impart color and/or other optical attributes to a cosmetic or personal care formulation. The materials described herein can be used as components separated or shielded by the compositions of the invention, and/or they may be used in a cosmetic formulation or personal care formulation to which the composition is added. For example, in addition to being used in the liquid compositions, the colorants described herein may be being dispersed throughout a suitable base or vehicle. For example, in the case of lipstick the coloring agent or pigmented ingredients may be dispersed in a base comprising a mixture of waxes (typically from 5-20% by weight), emollients, and moisturizers, whereas, in the case of blush, the pigmented solids may be dispersed in a base comprising a mixture of talc, kaolin, and various known binders.

The colorants used in the compositions may comprise pigments, which may be organic or inorganic. Exemplary inorganic pigments include, but are not limited to, inorganic oxides (e.g., metal oxides) and hydroxides such as magnesium oxide, magnesium hydroxide, calcium oxide, calcium hydroxides, aluminum oxide, aluminum hydroxide, silica, iron oxides (e.g., α-Fe₂O₃, γ-Fe₂O₃, Fe₃O₄, FeO) and iron hydroxides including red iron oxide, yellow iron oxide and black iron oxide, titanium dioxide, titanium lower oxides, zirconium oxide, chromium oxides, chromium hydroxides, manganese oxides, manganese hydroxides, cobalt oxides, cobalt hydroxides, cerium oxides, cerium hydroxides, nickel oxides, nickel hydroxides, zinc oxides and zinc hydroxides and composite oxides and composite hydroxides such as iron titanate, cobalt titanate and cobalt aluminate and the like. In one embodiment, the pigments have a particle size from 5 nm to 500 microns, or from 10 nm to 100 microns, or from 100 nm to 30 microns or from about 0.75 to 20 microns. In some embodiments, the particle size (median) will be less than about 10 microns, less than about 5 microns or less than 1 micron.

Other suitable colorants contemplated for use in the invention either as a component to be separated or shielded, or in the final cosmetic or personal care composition, are well known in the art, and are disclosed in the C.T.F.A. Cosmetic Ingredient Handbook, First Edition, 1988, the contents of which are hereby incorporated by reference. Lakes include, for example, FD&C lakes and D&C lakes. Lakes may include those based on barium, strontium, calcium or aluminum. Additional exemplary color additive lakes include, for example: D&C Red No. 19 (e.g., CI 45170, CI 73360 or CI 45430); D&C Red No. 9 (CI 15585); D&C Red No. 21 (CI 45380); D&C Orange No. 4 (CI 15510); D&C Orange No. 5 (CI 45370); D&C Red No. 27 (CI 45410); D&C Red No. 13 (CI 15630); D&C Red No. 7 (CI 15850:1); D&C Red No. 6 (CI 15850:2); D&C Yellow No. 5 (CI 19140); D&C Red No. 36 (CI 12085); D&C Orange No. 10 (CI 45475); D&C Yellow No. 19 (CI 15985); FD&C Red #40 (CI# 16035); FD&C Blue #1 (CI# 42090); FD&C Yellow #5 (CI# 19140); or any combinations thereof.

Additional suitable particulate colorants include carbon black, ultramarine blue, ferric blue, Prussian blue, manganese violet, talc, mica, sericite, calcium carbonate, fumed silica, and the like.

Pearlescents (pearling pigments) and interference pigments may also be used in the compositions of the invention. Suitable pearling pigments include without limitation, nacre, mica-based pearls, bismuth oxychloride, guanine and titanium composite materials containing, as a titanium component, titanium dioxide, titanium lower oxides or titanium oxynitride. Other suitable pearlescent materials typically are pigments or layers of titanium dioxide on a substrate such as mica, polyethylene terephthalate, bismuth oxychloride, aluminum oxide, calcium borosilicate, synthetic flourophlogopite (synthetic mica), silica, acrylates copolymer, methyl methacrylate, and the like. Pearlescents may also include mica particles at the surface of which are superposed at least two successive layers of metal oxides and/or of organic dyes.

Other suitable pearlescent agents, for example, include the (mica-based) pearlescent agents Timica, Flamenco and Duochrome sold by Engelhard, the Timiron pearlescent agents sold by Merck, the Prestige mica-based pearlescent agents sold by Eckart, and the Sunshine synthetic mica-based pearlescent agents sold by Sun Chemical.

Additional suitable pearlescents include, for example, the gold-colored pearlescent agents sold by Engelhard as Brillant Gold 212G (Timica), Gold 222C (Cloisonne), Sparkle gold (Timica), Gold 4504 (Chromalite) and Monarch gold 233X (Cloisonne); the bronze pearlescent agents sold by Merck as Bronze fine (17384) (Colorona) and Bronze (17353) (Colorona) and by Engelhard as Super bronze (Cloisonne); the orange pearlescent agents sold by Engelhard as Orange 363C (Cloisonne) and Orange MCR 101 (Cosmica) and by Merck as Passion orange (Colorona) and Matte orange (17449) (Microna); the brown-colored pearlescent agents sold by Engelhard as Nu-antique copper 340XB (Cloisonne) and Brown CL4509 (Chromalite); the pearlescent agents with a copper tint sold by Engelhard as Copper 340A (Timica); the pearlescent agents with a red tint sold by Merck as Sienna fine (17386) (Colorona); the pearlescent agents with a yellow tint sold by Engelhard as Yellow (4502) (Chromalite); the red-colored pearlescent agents with a gold tint sold by Engelhard as Sunstone G012 (Gemtone); the pink pearlescent agents sold by Engelhard as Tan opale G005 (Gemtone); the black pearlescent agents with a gold tint sold by Engelhard as Nu antique bronze 240 AB (Timica), the blue pearlescent agents sold by Merck as Matte blue (17433) (Microna), the white pearlescent agents with a silvery tint sold by Merck under as Xirona Silver and the golden-green pink-orange pearlescent agents sold by Merck as name Indian summer (Xirona); and mixtures thereof.

It may be desirable to employ surface modified pigments to adjust or improve dispersibility, water-resistance, oil-resistance, and the like. For example, pigments may be surface-modified with alkyl silane (e.g., caprylyl silane), Triethoxy Caprylylsilane or Triethoxy Caprylylsilane.

Suitable goniochromatic coloring agent may include, for example, multilayer interference structures and liquid-crystal coloring agents. Suitable symmetrical multilayer interference structures that may be used in the compositions of the invention include, for example, Al/SiO₂/Al/SiO₂/Al, pigments sold by Dupont de Nemours; Cr/MgF₂/Al/MgF₂/Cr pigments having sold as Chromaflair by Flex; Mo5₂/SiO₂/Al/SiO₂MoS₂;Fe₂O₃/SiO₂/Al/SiO₂/Fe₂O₃, and Fe₂O₃/SiO₂/Fe₂O₃/SiO₂/Fe₂O₃ pigments sold as Sicopearl by BASF; MoS2/SiO2/mica-oxide/SiO2/MoS2; Fe₂O₃/SiO₂/mica-oxide/SiO₂/Fe₂O₃; TiO₂/SiO₂/TiO₂ and TiO₂/Al₂O₃/TiO₂; SnO/TiO₂/SiO₂/TiO₂/SnO; Fe₂O₃/SiO₂/Fe₂O₃; SnO/mica/TiO₂/SiO₂/TiO₂/mica/SnO pigments sold as Xirona® by Merck (Darmstadt). These pigments may be, for example, pigments of silica/titanium oxide/tin oxide structure sold as Xirona Magic® by Merck, the pigments of silica/brown iron oxide structure sold as Xirona Indian Summer® by Merck, and the pigments of silica/titanium oxide/mica/tin oxide structure sold as Xirona Caribbean Blue® by Merck. Mention may also be made of the Infinite Colors® pigments from Shiseido. Depending on the thickness and the nature of the various layers used, different effects may be obtained. Suitable liquid-crystal goniochromatic particles include, for example, those sold by Chemx as well as those sold as Helicone® HC by Wacker.

Suitable fillers may include talc, silica, alumina, zinc stearate, mica, kaolin, nylon (in particular orgasol) powder, polyethylene powder, polypropylene powder, acrylates powders, Teflon, starch, boron nitride, copolymer microspheres such as Expancel (Nobel Industrie), Polytrap (Dow Coming), and silicone resin microbeads (Tospearl from Toshiba).

Other suitable fillers include inorganic powders such as chalk, fumed silica, fumed alumina, calcium oxide, calcium carbonate, magnesium oxide, magnesium carbonate, Fuller's earth, attapulgite, bentonite, muscovite, phlogopite, synthetic mica, lepidolite, hectorite, biotite, lithia mica, vermiculite, aluminum silicate, aluminum magnesium silicate, diatomaceous earth, starch, alkyl and/or trialkyl aryl ammonium smectites, chemically modified magnesium aluminum silicate, organically modified montmorillonite clay, hydrated aluminum silicate, hydrated silica, fumed aluminum starch octenyl succinate barium silicate, calcium silicate, magnesium silicate, strontium silicate, metal tungstate, magnesium, silica alumina, zeolite, barium sulfate, calcined calcium sulfate (calcined gypsum), calcium phosphate, fluorine apatite, hydroxyapatite, ceramic powder, metallic soap (zinc stearate, magnesium stearate, zinc myristate, calcium palmitate, and aluminum stearate), colloidal silicon dioxide; organic powder, cyclodextrin, methyl polymethacrylate powder, copolymer powder of styrene and acrylic acid, benzoguanamine resin powder, and poly(ethylene tetrafluoride) powder. Fillers may comprise from about 0.1% to about 20% by weight of the composition.

Sunscreen actives contemplated for use in the compositions of the invention may be organic or inorganic, and/or water-soluble or oil soluble, and include those with UVA and/or UVB absorbance from about 290 to about 400 nanometers solar radiation. Such sunscreen actives include, but are not limited to, one or more of the following: DEA methoxycinnamate, octylmethoxy cinnamate, drometrizole trisiloxane, oxybenzone, octyl methoxycinnamate, octyl salicylate, homomenthyl salicylate, octocrylene, avobenzone, octyl dimethyl PABA, TEA salicylate, 4-methyl benzilidene camphor, terephthalydiene dicamphor sulfonic acid, ethyl PABA, hydroxy methylphenyl benzotriazole, methylene bis-benzotriazoyltetramethylbutylphenol, bis-ethylhexyloxyphenol methoxyphenol triazine, zinc oxide, titanium dioxide or any derivatives, or any combinations thereof. The sunscreen may be present, for example, from about 1% by weight to about 30% by weight of the total weight of the composition.

The cosmetic compositions and personal care products of the invention may also comprise any conventional components, including fillers and cosmetic powders, film forming polymers, gelling agents, waxes, thickeners, conditioners, actives, solvents, emulsifiers, humectants, emollients, pH adjusters, antioxidants, preservatives, fragrances, and the like. Such components may be present, for example, in an individual or collective amount from about 0.1% to about 30% by weight.

The gelling agent may comprise, for example, one or more of a silicone resin, including Dimethicone/Vinyl Dimethicone crosspolymer, silicone T-resins, ETPEA, polyamides, cellulose ethers (e.g., methyl cellulose or ethyl cellulose) and the like. Thickeners such as acrylates copolymers, hydroxyalkyl cellulose, carboxymethylcellulose, carbomers, and vegetable gums such as xanthan gum may be included.

The compositions may include natural or synthetic film-forming polymers. Suitable polymeric film formers include polyolefins, silicone polymers (e.g., dimethicones, dimethiconols, amodimethicones, silicone resins, etc.), (meth)acrylates, alkyl (meth)acrylates, polyurethanes, fluoropolymers, silicone polyurethanes, and silicone acrylates such as acrylates/dimethicone copolymers. In some embodiments, it may be desirable to add a hydrophilic or water-soluble film former (e.g., cellulosics, polysaccharides, polyquaterniums (such as polyquaternium-37 (INCI), etc.) to the composition to improve spreading, emulsion stability, aesthetic look and feel, etc. Elastomers formed from ethylene, propylene, butylene, and/or styrene monomers may also be useful. Firm forming polymers may be present from about 0.1% to about 10% by weight of the composition.

Suitable emollients include, without limitation, isopropyl myristate, petrolatum, volatile or non-volatile silicones oils (e.g., methicone, dimethicone), ester oils, mineral oils, hydrocarbon oils, and fatty acid esters. Emollients may comprise from about 0.1% to about 50% by weight of the composition.

Suitable humectants such as polyols (e.g., glycols), including without limitation, glycerin, propylene glycol, ethoxydiglycol, butylene glycol, pentylene glycol, hexylene glycol, caprylyl glycol, and the like. These will typically be added in amount from about 0.001 to about 5% by weight. Humectants, if present, may comprise from about 0.1% to about 50% by weight of the composition.

In another embodiment, the compositions of the invention may also include one or more of the following: a skin penetration enhancer; a skin plumper, such as palmitoyl oligopeptide, collagen, collagen and/or glycosaminoglycan (GAG) enhancing agents; an exfoliating agent; and an antioxidant (e.g., TDPA).

Suitable waxes that may be used alone or in combination include, without limitation, natural waxes, mineral waxes, and synthetic waxes. Natural waxes are those of animal origin, including, without limitation, beeswax, spermaceti, lanolin, and shellac wax, and those of vegetable origin, including, without limitation, carnauba, candelilla, bayberry, and sugarcane wax. Special mention may be made of silicone wax.

Mineral waxes contemplated to be useful include, without limitation, ozokerite, ceresin, montan, paraffin, microcrystalline, petroleum, and petrolatum waxes.

Suitable synthetic waxes which may be used in a final cosmetic product formulation or personal care product formulation (but not for use as a polymeric thickener in the multi-component composition of the invention) include, for example, polyethylene glycols such as PEG-18, PEG-20, PEG-32, PEG-75, PEG-90, PEG-100, and PEG-180 which are sold under the tradename CARBOWAX® (The Dow Chemical Company). Carbowax 1000 has a molecular weight range of 950 to 1,050 and a melting point of about 38° C., Carbowax 1450 has a molecular weight range of about 1,305 to 1,595 and a melting point of about 56° C., Carbowax 3350 has a molecular weight range of 3,015 to 3,685 and a melting point of about 56° C., and Carbowax 8000 has a molecular weight range of 7,000 to 9,000 and a melting point of about 61° C.

Additional suitable synthetic waxes include Fischer Tropsch (FT) waxes and polyolefin waxes, such as ethylene homopolymers, ethylene-propylene copolymers, and ethylene-hexene copolymers. Representative ethylene homopolymer waxes are commercially available under the tradename POLYWAX® Polyethylene (Baker Hughes Incorporated) with melting points ranging from 80° C. to 132° C. Commercially available ethylene-α-olefin copolymer waxes include those sold under the tradename PETROLITE® Copolymers (Baker Hughes Incorporated) with melting points ranging from 95° C. to 115° C.

Waxes may individually or collectively comprise from about 0.1% to about 30% by weight of the composition.

Other suitable materials for use in the invention may include skin benefit agents such as antioxidants (e.g., BHT, ascorbic acid, sodium ascorbate, ascorbyl palmitate, beta-carotene, etc.), vitamins (e.g., tocopherol, tocopheryl acetate, etc.), alpha-hydroxy acids (e.g., glycolic acid), beta-hydroxy acids (e.g., salicylic acid), retinoids (e.g., retinoic acid, all-trans-retinoic acid, retinaldehyde, retinol, and retinol esters such as acetates or palmitates), other anti-aging ingredients (e.g., collagen stimulators, collagenase inhibitors, elastase inhibitors), depigmenting agents (e.g., TDPA, hydroquinone, kojic acid), barrier function enhancing agents (e.g., ceramides, glycerides, cholesterol and its esters, alpha-hydroxy and omega-hydroxy fatty acids and esters thereof, etc.), exfoliating agents, estrogen synthetase stimulating compounds (e.g., caffeine and derivatives), compounds capable of inhibiting 5 alpha-reductase activity (e.g., linolenic acid, linoleic acid, finasteride, and mixtures thereof). These benefit agents will typically be present, if at all, in amounts between about 0.001% and about 10% by weight of the composition.

Additional suitable materials may include botanicals, keratolytic agents, keratinocyte proliferation enhancers, anti-inflammatory agents, steroids, desthiobiotin, piperazine carboxamide, cis-6-nonenol, arginine, glucosamine, algae extract, chlorphenesin, advanced glycation end-product (AGE) inhibitors, and PLOD-2 stimulators (e.g., N-acetyl amino acid amides, such as N-Acetyl Tyrosinamide).

The compositions may further include an emulsifier. The amount of emulsifier will typically be from about 0.001 to about 10% by weight, but preferably will range from about 0.01 to about 5% by weight, and most preferably about 0.1 to about 1% by weight, based upon the total weight of the composition. The emulsifier may be ionic, zwitterionic, or nonionic. Suitable emulsifiers include those of the polyethoxylated type (e.g., polyoxyethylene ethers or esters), polydiorganosiloxane-polyoxyalkylene block copolymers (e.g., dimethicone copolyol), Steareth-20, Steareth-21, fatty alcohols (e.g., Cetearyl Alcohol), Polyoxethylene sorbitan fatty acid esters (i.e., polysorbates), and Hydrogenated Castor Oil, to name a few. Additional emulsifiers are provided in the INCI Ingredient Dictionary and Handbook 11 th Edition 2006, the disclosure of which is hereby incorporated by reference.

The compositions may comprise a cationic polymer. Cationic polymers include, but are not limited to, polyquaternium 4, polyquaternium 6, polyquaternium 7, polyquaternium 10, polyquaternium 11, polyquaternium 16, polyquaternium 22, polyquaternium 28, polyquaternium 32, and guar hydroxypropyltrimonium chloride. When present, the cationic polymer will typically comprise an amount of about 0.1% to about 15% by weight of the composition. In other embodiments the compositions may contain an amount of cationic (quaternium) ingredients that are anhydrous or have very low level of water, e.g., less than 1% by weight. Other suitable quaternium compounds include, without limitation, Polyquaternium-37 (INCI), Silicone Quaternium-18 (INCI), PEG-2 Dimeadowfoamamidoethylmonium Methosulfate and Hexylene Glycol (INCI), and Cetrimonium Chloride (INCI), to name a few. Such quaternium compounds, if present, will typically comprise from about 0.05% to about 5% by weight of the total composition, and more typically, from about 0.1% to about 1.5% by weight.

The compositions may also comprise monomer quaternary ammonium compounds such as, for example, alkyltrimethylammonium chlorides, dialkylmethyl-ammonium chlorides, alkyldimethylbenzylammonium chlorides, and alkylpyridinium chlorides. In one embodiment, the composition comprises at least one conditioning agent selected from the group consisting of polyquaterniums, cationic polymers, cationic surfactants, non-volatile dimethicone oils, dimethiconols, amodimethicones, ester oils, fatty alcohols, cationic gums and cellulosics, amido amines, cetrimonium chloride, behentrimonium chloride, stearamidopropyl dimethylamine, polyesteramines, and cationically charge-modified polymers derived from guar gum, cellulose, proteins, polypeptides, chitosan, lanolin, starches and amino silicones.

The compositions may include a nonionic surfactant such as Laureth-23, Ceteth-10, Ceteth-20, IsoCeteth-20, Steareth-20, Oleth-10, Oleth-20, or alkyl polyglucose. The nonionic surfactant may be formed from a fatty alcohol, a fatty acid, or a glyceride with a C8 to C24 carbon chain. The compositions of the invention can further comprise proteins, peptides, and amino acids including hydrolyzed soy protein, lauryldimonium hydrolyzed soy protein (cationic Soya protein), wheat amino acids, corn, wheat, milk, or silk proteins, collagens, keratins, taurine and arginine hydrochloride, etc.

The cosmetic compositions of the invention may optionally include one or more agents that provide or enhance shine on a keratin fiber. Shine enhancing agents will typically have a refractive index greater than about 1.4, preferably greater than about 1.5 when measured as a film at 25° C. Suitable shine enhancing agents include without limitation, polyols, fatty esters, silicone phenylpropyldimethylsiloxysilicate, polybutene, polyisobutene, hydrogenated polyisobutene, hydrogenated polycyclopentadiene, propyl phenyl silsesquioxane resins; lauryl methicone copolyol, perfluorononyl dimethicone, dimethicone/trisiloxane, methyl trimethicone, and combinations thereof. In one embodiment, the composition will comprise a shine-enhancing agent in an amount from about 0.1% to about 10% by weight, based on the total weight of the composition.

The compositions may also comprise a preservative or anti-microbial agent, for example, methylchloroisothiazolinone, methylisothiazolinone, methylparaben, propylparaben, phenoxyethanol, or caprylyl glycol.

The compositions of the invention may include a cosmetically or dermatologically acceptable vehicle that is substantially anhydrous. As used herein, “substantially anhydrous” means comprising less than 5% water. In other embodiments, the vehicle and/or the entire cosmetic or personal care composition comprises less than 4%, less than 3%, less than 2%, less than 1%, less than 0.5%, or less than 0.1% by weight water.

The vehicle may be in the form of, for example, a serum, a cream, a lotion, a gel, or a stick, and may comprise an emulsion (e.g., polyol-in-silicone, silicone-in-polyol emulsion, etc.), or may comprise an ethanolic vehicle, silicone (e.g., cyclomethicone, dimethicone, etc.), hydrocarbon (e.g., petrolatum, isododecane, etc.), ester oil (e.g., isopropyl myristate, myristyl myristate), or the like. The vehicle may further comprise an emulsifier, gelling agent, structuring agent, rheology modifier (e.g., a thickener), film former, or the like. The vehicle may comprise from about 25% to about 99% by weight of the composition.

In another aspect of the invention, methods are provided for delivering one or more colorants in a single formulation. In some embodiments, methods are provided for delivering in a single formulation, one or more colorants or other ingredients that can impart color or other optical properties (e.g., hue value, chroma, color travel, interference, etc.) to a cosmetic or personal care formulation. The method comprises applying to a human integument a composition of the invention, comprising a first liquid composition (continuous phase) that comprises water and a laponite clay, and a second liquid composition (discontinuous phase) that comprises water and a polymeric thickener. The compositions are applied to a human integument for example, by the application of, for example, pressure or shear, such as by rubbing, blotting, or patting, etc. Application of the composition can release the contents of the discontinuous phase into the continuous phase, so that the contents of the discontinuous phase can blend together with the continuous phase, providing a substantially uniform appearance. The amount of pressure applied during rubbing and the duration of rubbing needed will vary based on the particular components and, for example, the desired look, shine, color, or texture. In some embodiments, the contents of discontinuous phase are not released into the continuous phase, and do not blend with the continuous phase, thereby providing an inhomogeneous color deposit on an integument.

The compositions of the invention may be applied to the human integumentary system, including, for example, skin, lips, nails, hair, and other keratinous surfaces. As used herein, the term “keratinous surface” refers to keratin-containing portions of the human integumentary system, which includes, but is not limited to, skin, lips, hair (including hair of the scalp, eyelashes, eyebrows, facial hair, and body hair such as hair of the arms, legs, etc.), and nails (toenails, fingernails, cuticles, etc.) of mammalians, preferably humans.

The compositions of the invention can be applied to any area of the skin, and preferably to the face, the neck, the hands, the feet, or other areas of the body, such as arms, legs, and back.

In one embodiment, the composition is intended for use as a non-therapeutic treatment. In another embodiment, the composition is an article intended to be rubbed, poured, sprinkled, or sprayed on, introduced into, or otherwise applied to the human body for cleansing, beautifying, promoting attractiveness, or altering the appearance, in accordance with the US FD&C Act, §201(i).

### EXAMPLES

### Example 1

### Multi-Colored Composition

A composition comprising multiple shades of the same pigments segregated into separate phases was prepared according to the methods of the invention. The first liquid composition (continuous phase) was prepared according to the formula in Table 1 below.

TABLE 1
Continuous phase

| Ingredient | % |
|---|---|
| Water | 90.35 |
| Laponite S482 | 8.04 |
| Optigel | 1.61 |
| Total | 100 |

The laponite clay (Rockwood Additives) and water were mixed with a propeller blade for 20-30 minutes, until the mixture was clear and the laponite clay fully dispersed. Optigel (BYK Additives and Instruments) was then added and mixed until fully dispersed.

A second, third, and fourth liquid composition (discontinuous phases; labeled as liquids 2-4 in Table 2 below) were prepared, each made with the same combination of pigments, but each having a different shade (liquid 2 is light shade; liquid 3 is dark shade, and liquid 4 is white) based on the amount of each pigment used, according to the formula in Table 2 below.

TABLE 2
Discontinuous phases

| | Liquid 2 | Liquid 3 | Liquid 4 |
|---|---|---|---|
| Ingredient | (%) | (%) | (%) |
| Water | 94.1 | 94.1 | 94.1 |
| Hydroxyethyl | 0.9 | 0.9 | 0.9 |
| cellulose | | | |
| Titanium dioxide | 4.25 | 3.19 | 5 |
| Yellow iron | 0.54 | 1.26 | — |
| oxide | | | |
| Red iron oxide | 0.11 | 0.23 | — |
| Black iron oxid | 0.1 | 0.32 | — |
| Total | 100 | 100 | 100 |

The water and hydroxyethyl cellulose were mixed with a propeller blade and with slight heat until fully dispersed, and the liquid was slightly hazy. The pigment was added and the mixture was milled for about 10 minutes at about 4,000 rpm.

The discontinuous phases were then added to the continuous phase, while the continuous phase was being mixed with a propeller blade. The amount of each shade (each discontinuous phase) included in the final formulation is shown in Table 3 below.

TABLE 3
Multi-colored composition

| Ingredient | % |
|---|---|
| Continuous phase | 62.2 |
| Discontinuous phase | |
| Liquid 2 | 9.45 |
| Liquid 3 | 9.45 |
| Liquid 4 | 18.90 |
| Total | 100 |

The resulting composition maintained separation of the different shades and did not blend together. The appearance of the composition was speckled and multi-colored.

### Example 2

### Multi-Colored Foundation Stick

A multi-colored solid foundation stick comprising the composition of Table 3 was prepared according to the methods of the invention. The formulation for the solid stick is shown below in Table 4.

TABLE 4

| Ingredient | % |
|---|---|
| Deinoized water | 76.15 |
| Glycerin | 2.00 |
| SeaPure ™ Agarose | 3.50 |
| Hydroxyethyl cellulose | 3.25 |
| Hydrogenated Castor Oil | 5.00 |
| Hyaluronic Acid | 0.10 |
| Composition of Table 3 | 10.00 |
| Total | 100 |

The water was added to a beaker, and mixed with a propeller blade. Glycerin was added to the water, and mixed for five minutes, before the SeaPure™ agarose was added, and the mixture was heated to 90° C. Mixing was continued until hazy/clear. One the agarose is fully dispersed, the Hydroxyethyl cellulose was added and dispersed completed. Hyaluronic acid and castor oil were then added and mixed until dispersed. Next, the composition of Table 3 was added to the mixture, and mixed until uniform. The mixture was then cooled to 50° C. with propeller mixing, before removed from the heat and liquid composition was poured into lipstick molds and cooled to 10° C. to form sticks.

The resulting multi-color foundation stick had a speckled appearance, maintaining separation of the different shades.

### Example 3

### Multi-Colored Foundation Stick

An additional multi-colored solid foundation stick was prepared according to the methods of the invention, comprising the composition of Table 3. The formulation for the solid stick is shown below in Table 5.

TABLE 5

| Ingredient | % |
|---|---|
| Deionized water | 43.20 |
| Algin | 0.50 |
| Butylene glycol | 25.00 |
| Allantoin | 0.20 |
| Xanthan Gum | 0.10 |
| Kaolin | 2.00 |
| POE (20M) sorbitan monolaurate | 0.15 |
| Sodium stearate | 2.50 |
| Sodium chloride | 0.25 |
| Benzyl alcohol | 1.00 |
| Potassium hydroxide | 0.10 |
| Composition of Table 3 | 25.00 |
| Total | 100 |

The water was heated to 60-63° C., and algin was added and mixed for about 30-45 minutes with a propeller blade until completely dispersed. Butylene glycol and allantoin were added and mixed for 10 minutes, until the mixture was uniform. Xanthan gum was then added, after which kaolin was added and then mixed until uniform. The mixture was then heated to about 77-79° C. POE (20M) sorbitan monolaurate was added and mixed for 5 minutes. Sodium stearate was then added and mixed for about 45 minutes. Sodium chloride was mixed on slow speed, after which the composition of Table 3 was added, and mixed about 2-3 minutes. The liquid mixture was then removed from the heat and was poured into lipstick molds and cooled to 10° C. to form sticks.

The resulting multi-color foundation stick had a speckled appearance, maintaining separation of the different shades that did not blend together, both prior to application, and after application of the foundation stick to the skin.

### Example 4

### Blendable Multi-Colored Foundation Stick

An additional multi-colored solid foundation stick was prepared according to the methods of the invention, comprising the composition of Table 3. The foundation stick in this example included an outer core comprising the multi-colored solid stick foundation described in Example 3 and an inner core that comprised PEG and the other ingredients listed below in Table 6.

TABLE 6

| Ingredient | % |
|---|---|
| Deinoized water | 43.10 |
| Algin | 0.50 |
| Propylene glycol | 5.00 |
| Butylene glycol | 25.00 |
| Allantoin | 0.20 |
| Xanthan Gum | 0.20 |
| Kaolin | 2.00 |
| POE (20M) sorbitan monolaurate | 0.15 |
| Sodium stearate | 2.50 |
| Sodium chloride | 0.25 |
| Benzyl alcohol | 1.00 |
| Potassium hydroxide | 0.10 |
| Polyethylene glycol-40 | 10.00 |
| Polyethylene glycol-12 caranuba wax | 10.00 |
| Total | 100 |

The resulting multi-color foundation stick had a speckled appearance, maintaining separation of the different shades that did not blend together prior to application to the skin. In addition, upon application of the foundation stick to the skin, the different shades that were segregated in the composition became blended, resulting in a different, uniform color on the skin.

The invention described and claimed herein is not to be limited in scope by the specific embodiments herein disclosed since these embodiments are intended as illustrations of several aspects of the invention. Any equivalent embodiments are intended to be within the scope of this invention. Indeed, various modifications of the invention in addition to those shown and described therein will become apparent to those skilled in the art from the foregoing description. Such modifications are also intended to fall within the scope of the appended claims. All publications cited herein are incorporated by reference in their entirety.

What is claimed:

1. A method of making a composition capable of maintaining separation between two or more liquids having different optical attributes, the method comprising:
mixing together at least:
(a) a first liquid composition comprising water and a laponite clay;
(b) a second liquid composition comprising water and a polymeric thickener; and
(c) a third liquid composition comprising water and a polymeric thickener;
wherein said first, second, and third liquid compositions are separated such that the interaction between them is reduced compared to an otherwise identical composition lacking the laponite clay;
and wherein at least two of said first, second, and third liquid compositions have different optical attributes.

2. The method according to claim 1 wherein the laponite clay comprises sodium magnesium fluorosilicate or sodium magnesium silicate.

3. The method according to claim 1 wherein the laponite clay comprises from about 5% to about 8% by weight of the composition.

4. The method according to claim 1 wherein the polymeric thickener comprises hydroxyethylcellulose.

5. The method according to claim 1 wherein the polymeric thickener comprises from about 0.1 to about 10% by weight of the composition.

6. The method according to claim 1 wherein the first, second, and third liquid compositions each have different optical properties.

7. The method according to claim 1 wherein the first, second, and third liquid compositions each comprise a colorant.

8. The method according to claim 7 wherein at least two of the first, second, and third liquid compositions comprise different colorants.

9. The method according to claim 7 wherein the colorants comprise pigments.

10. The method according to claim 7 wherein at least two of the first, second, and third liquid compositions comprise different levels of a same colorant.

11. The method according to claim 1 wherein the second and third liquid compositions each comprise a colorant.

12. The method according to claim 11 wherein the second and third liquid compositions each comprise different colorants.

15. The method according to claim 1 wherein the composition is in the form of a cream, a lotion, or a solid stick.

14. A composition capable of maintaining separation between two or more liquids having different optical attributes, the composition comprising:
(a) a first liquid composition comprising water and a laponite clay;
(b) a second liquid composition comprising water and a polymeric thickener; and
(c) a third liquid composition comprising water and a polymeric thickener;
wherein said first, second, and third liquid compositions are separated such that the interaction between them is reduced compared to an otherwise identical composition lacking the laponite clay;
and wherein at least two of said first, second, and third liquid compositions have different optical attributes.

15. A method of delivering two or more components to a human integument comprising:
applying to said human integument a composition comprising:
(a) a first liquid composition comprising water and a laponite clay;
(b) a second liquid composition comprising water and a polymeric thickener; and
(c) a third liquid composition comprising water and a polymeric thickener;
wherein said first, second, and third liquid compositions are separated such that the interaction between them is reduced compared to an otherwise identical composition lacking the laponite clay;
and wherein at least two of said first, second, and third liquid compositions have different optical attributes.

16. The method according to claim 15, wherein at least one of the first, second, and third liquid compositions comprises a poly(alkene oxide), and further comprising the step of applying shear to said composition after application to said human integument to release the contents of the second and third liquid compositions.

17. The method according to claim 16, wherein said poly(alkene oxide) comprises polyethylene glycol.

18. The method according to claim 17, wherein said polyethylene glycol comprises PEG-12 carnauba wax.

19. The method according to claim 17, wherein said polyethylene glycol comprises PEG-40.

20. The method acceding to claim 16, wherein said composition changes color upon application of shear.
